(19) United States
(12) Patent Application Publication
(10) Pub. No.: US 2007/0048863 A1
(43) Pub. Date: Mar. 1, 2007

(54) Computerized factorial experimental design and control of reaction sites and arrays thereof

(75) Inventors: Seth T. Rodgers, Somerville, MA (US); Fan Zhang, Lexington, MA (US); Mohamed Shaheen, Methuen, MA (US); Benjamin Russell Alexander, Somerville, MA (US)

Correspondence Address: WOLF GREENFIELD & SACKS, PC, FEDERAL RESERVE PLAZA, 600 ATLANTIC AVENUE, BOSTON, MA, 02210-2206, US

(73) Assignee: BioProcessors Corp., Woburn, MA (US)

(21) Appl. No.: 11/492,445

(22) Filed: Jul. 25, 2006

Related U.S. Application Data
(60) Provisional application No. 60/702,308, filed on Jul. 25, 2005.
(60) Provisional application No. 60/774,426, filed on Feb. 17, 2006.

(51) Int. Cl. C12N 5/06; G06F 19/00

(52) U.S. Cl. 435/325; 702/19

(57) ABSTRACT

Computer-facilitated design of large-scale, multi-factorial cell culture experiments and the like, and control of reaction sites and/or arrays of reaction sites to perform such experiments using automated devices. In certain cases, the invention is directed to controlling a plurality of cell culture experiments, e.g., using an automated cell culture device. In one set of embodiments, a data structure or a “descriptor” for use with cell culture experiments is provided. The descriptor may be used, for instance, to control one or more cell culture experiments, to identify one or more cell culture experiments, and/or to identify or “tag” data arising from one or more cell culture experiments, e.g., for further analysis or recall.

### CROSS-REFERENCE TO RELATED APPLICATIONS

This application claims the benefit, under 35 USC 119(e) of prior U.S. provisional patent applications Nos. 60/702,308, filed 25 Jul. 2005, CONTROL OF REACTORS INCLUDING COMPUTER IMPLEMENTATIONS; and 60/774,426, filed 17 Feb. 2006, titled COMPUTERIZED FACTORIAL EXPERIMENTAL DESIGN AND CONTROL OF REACTION SITES AND ARRAYS THEREOF.

### FIELD OF INVENTION

The present invention generally relates to the computer-facilitated design of large-scale, multi-factorial cell culture experiments and the like, and to the control of reaction sites and/or arrays of reaction sites to perform such experiments using automated devices; and to indexed search and analysis of experimental results thereby obtained.

### BACKGROUND

Cells are cultured for a variety of reasons. Increasingly, cells are cultured for proteins or other valuable materials they produce. Many cells require specific conditions, such as a controlled environment. The presence of nutrients, metabolic gases such as oxygen and/or carbon dioxide, humidity, as well as other factors such as temperature, may affect cell growth and/or cellular product expression. Cells require time to grow, during which the environmental conditions to which the cells are exposed will influence the biochemical behavior of the cells, such as whether the cells express certain proteins or do not express those proteins, and the quantity or composition (mix) of such product(s). In some cases, such as with particular bacterial cells, a successful cell culture may be performed in as little as 24 hours. In other cases, such as with particular mammalian cells, a successful culture may require about 30 days or more.

Typically, cell culture experiments are performed in various types of media containing necessary nutrients, for example, glucose, glutamine, pyruvate, and/or various amino acids, vitamins, hormones, serum, ions, or the like. The cells are generally cultured in a location, such as an incubator, where the environmental conditions can be controlled, for example, the temperature, O₂ and/or CO₂ concentration, relative humidity, etc. Recently, as described in International Patent Application No. PCT/US01/07679, filed Mar. 9, 2001, entitled “Microreactor,” by Jury, et al., published as WO 01/68257 on Sep. 20, 2001, incorporated herein by reference, cells have also been cultured on a very small scale (i.e., on the order of a few milliliters or less), so that, among other things, many cultures can be performed in parallel.

In general, the current approaches to designing, setting up, and running cell culture experiments involve a significant amount of time and labor. For example, configuring the incubator and setting up the incubator controls for a single cell culture experiment may require multiple hours of a scientist or a technician's time. This “overhead” constrains the number and cost of most researchers' cell culture experiments. It is difficult to predict the conditions that will be effective or optimal for a given cell strain to produce a desired product, or whether it will do so at all or with a desired quantity or purity. Consequently, an experimenter often would like to be able to perform far more experiments than he or she conventionally is able or permitted to perform, due to cost and/or time constraints.

Moreover, if researchers were able to perform a significantly greater number of cell culture experiments without greater, or even with lesser cost, the public would receive the benefits of greater knowledge, potentially lower drug discovery cost, increased rate of drug discovery, etc. At the same time, challenges would present themselves in areas such as mining the large amount of resulting data, and avoiding costly duplication or execution of overlapping experiments.

Researchers also, for the most part, lack institutional memory of experiments that others in their organizations, much less others in other organizations. Hence, they may conduct experiments that have already been done, wasting resources and valuable time. Some of these experiments may take hundreds of hours, so that a researcher may lose weeks to unnecessary experiments. In races to identify new drugs and be the first to market them, such losses of time are highly undesirable.

In some instances, experiments are not done simply because an organization lacks the human capital to perform them.

Accordingly, needs exist for tools that will allow researchers to conduct more experiments, that allow researchers to perform more experiments in parallel and that allow an organization to perform more experiments without a concomitant increase in human lab workers. Needs further exist for tools that will allow researchers to share at least some experimental designs and results, whether intra- or inter-institutionally.

In order to facilitate the performance of larger scale biological experiments such as those discussed above, without huge numbers of scientists and/or technicians, needs exist both for robotic experimentation systems and for methods and systems usable by researchers to design and conduct such experiments using such robotic systems.

### SUMMARY OF THE INVENTION

The present invention generally relates to the computer-facilitated design of large-scale, multi-factorial cell culture experiments and the like, and to the control of reaction sites and/or arrays of reaction sites to perform such experiments using automated devices. The subject matter of the present invention involves, in some cases, interrelated products, alternative solutions to a particular problem, and/or a plurality of different uses of one or more systems and/or articles.

In one aspect, the invention is a method. The method, in one set of embodiments, includes acts of culturing a plurality of cell cultures in an automated cell culture device; for each cell culture, operating the automated cell culture device to collect data representative of a plurality of experimental factors of the cell culture at a plurality of times, which may be different for different cultures; and from the automated cell culture device, automatically recording the data in a data structure in a computer-readable store, that data structure comprising, for each culture, fields in a compute-readable memory defining a matrix in two dimensions, a first dimension representing experimental factors and a second dimension representing time events. In another set of embodiments, the method includes acts of culturing a plurality of cell cultures using an automated cell culture device. For each cell culture, the method also includes collecting data representative of a plurality of experimental factors of the cell culture at a plurality of times, and recording the data in a data structure in a data store. In some cases, the data structure comprises, for each culture, fields defining a matrix addressable in two dimensions, a first dimension representing experimental factors and a second dimension representing time events

The method, in another set of embodiments, includes acts of defining a plurality of experimental factors for a cell culture; for each experimental factor, defining a plurality of levels; generating a plurality of experimental protocols, using, for each factor, a respective level selected from the plurality of levels; and for each experimental protocol, applying the experimental protocol to a cell culture using an automated cell culture device.

The method, according to still another set of embodiments, includes acts of providing a plurality of reactor arrays, each comprising a plurality of reactors; defining at least one reaction factor that independently is selectable to operates on each reactor; defining at least one array factor that simultaneously, but not independently, operates on each reactor within a reactor array; for each reaction factor and each array factor, defining a plurality of levels; generating a plurality of experimental protocols using, for each reaction factor and each array factor, a respective level selected from the plurality of levels, where at least one protocol includes at least one reactor array that comprises a first reactor operated on by a first level of a reaction factor, and a second reactor operated on by a second level of the reaction factor; and for each protocol, applying the protocol to the reactor array using an automated device.

According to another set of embodiments, the method includes acts of providing a plurality of reactor arrays, each comprising a plurality of reactors; defining at least one reaction factor that may, if selected in an experimental protocol, independently operate on a selected reactor; defining at least one array factor that simultaneously, but not independently, operates on each reactor within a single reactor array; for each reaction factor and each array factor, defining a plurality of levels; and generating one or more sets of experimental protocols, applicable to the plurality of reactor arrays, such that at least one set of the one or more sets includes (1) a first experimental protocol applicable to a first reactor of a particular reactor array selected from the plurality of reactor arrays, and (2) a second experimental protocol applicable to a second reactor of the particular reactor array. In some cases, the first experimental protocol and the second experimental protocol each are generated using, for each selected reaction factor and each selected array factor, a respective level selected from the plurality of levels, where the first experimental protocol includes a first level of a reaction factor, and the second experimental protocol includes a second level of a reaction factor that is different from the first level. The method may also include an act of applying the set of experimental protocols to the corresponding particular reactor array using an automated device.

In another set of embodiments, the method includes acts of generating a plurality of cell culture protocols using a factorial design; culturing a plurality of cell cultures, using the plurality of cell culture protocols, in an automated cell culture device; collecting data from the plurality of cell cultures; and recording the data on a machine-readable medium in a data structure comprising a matrix representation of the factorial design, the matrix representation representing the values of the factors for each individual experiment as a collection of deviations from baseline conditions.

In yet another set of embodiments, the method is a computer-implemented method for use in performing cell culture experiments. The method comprising acts of, operating a computer to: present to a user an interface for receiving a set of experimental factors, one or more levels for each factor, and one or more times at which one or more factor values are to be set and/or one or more measurements are to be taken, receive as input from the user the set of experimental factors and the one or more times; create a data structure defining one or more experiments, using factorial design, comprising one or more experimental factors and one or more times; and assign each experimental protocol to a specific cell culture in a reactor array.

The method, in still another aspect, is a computer-implemented method, comprising acts of defining a plurality of elements and a plurality of groups containing the plurality of elements, at least one group containing more than one element; defining at least one elemental factor and at least one group factor, where each elemental factor independently operates on each element, and each group factor simultaneously, but not independently, operates on each element within a group; for each elemental factor and each group factor, defining a plurality of levels; and generating a plurality of protocols to operate on each group and each element within each group, using, for each elemental factor and each group factor, a respective level selected from the plurality of levels, where at least one protocol includes at least one group containing at least a first element and a second element where the first element is operated on by a first level of an elemental factor, and the second element is operated on by a second level of the elemental factor.

In yet another aspect, the method is a computer-implemented method, which comprises an act of operating a computer to prompt a user to input a plurality of groups, each comprising a plurality of elements, at least one group containing more than one element; at least one reaction factor that may, if selected in an experimental protocol, independently operate on a selected element; at least one group factor that simultaneously, but not independently, operates on each element within a single group; and, for each elemental factor and each group factor, a plurality of levels. The computer-implemented method also comprises an act of operating a computer to generate one or more sets of protocols applicable to the plurality of groups such that at least one set of the one or more sets includes (1) a first protocol applicable to a first element of a particular group selected from the plurality of groups, and (2) a second protocol applicable to a second element of the particular group. In some cases, the first protocol and the second protocol each can be generated using, for each selected elemental factor and each selected group factor, a respective level selected from the plurality of levels, where the first protocol includes a first level of an elemental factor, and the second protocol includes a second level of an elemental factor that is different from the first level. In certain instances, the method also includes acts of operating a computer to issue the one or more sets of protocols to an automated device, and cause the automated device to apply the one or more sets of protocols to an experimental system comprising a plurality of discrete experiments.

In yet another aspect, the method is a computer-implemented method for use in performing cell culture experiments. The method includes an act of presenting to a user an interface for receiving a list of experimental factors, one or more levels for each experimental factor, and one or more times at which one or more experimental factor values are to be set and/or one or more measurements are to be taken; receiving, as input, the list of experimental factors and the one or more times; creating a data structure corresponding to the received one or more experimental protocols, using the received factorial design, where each experimental protocol comprises one or more values for the experimental factors and one or more times; and assigning each experimental protocol to a specific cell culture contained in a reactor array.

The invention includes a system in another aspect. In one set of embodiments, the system includes an automated cell culture device comprising a machine-readable medium having stored thereon at least one data structure comprising fields defining a matrix in two dimensions. In some cases, a first dimension represents experimental factors and a second dimension represents time events.

Various articles are provided according to yet another aspect of the invention. In one set of embodiments, the article includes a machine-readable medium having stored thereon signals comprising instructions and at least one data structure for use in operating an automated cell culture device. In some embodiments, the data structure comprises fields defining a matrix in two dimensions, where a first dimension represents experimental factors and a second dimension represents time events.

The article, in some embodiments, comprises a machine-readable medium having stored thereon signals comprising instructions and at least one data structure for use in operating an automated cell culture device to cause the device to implement a series of cell culture experiments and to collect data therefrom. In some cases, the data structure comprises a matrix representation of a factorial design, where the matrix representation represents the values of the factors for each individual experiment as a collection of deviations from baseline conditions.

In some embodiments, the article is a machine-readable medium having a program stored thereon. In one embodiment, the program comprises instructions for, when executed, performing acts of receiving user input defining a plurality of factors representing experimental parameters for a cell culture experiment; for each factor, defining a plurality of levels for use in experiments; generating a plurality of protocols, using, for each factor, a respective level selected from the plurality of levels; and for each protocol, applying the protocol to a cell culture. In another embodiment, the program comprises instructions for, when executed, performing acts of defining a plurality of experimental factors for a cell culture; for at least some experimental factors, defining a plurality of levels; generating a plurality of experimental protocols, using, for each factor, a respective level selected from the plurality of levels; and, for each experimental protocol, applying the experimental protocol to a cell culture using an automated cell culture device.

In yet another aspect, the program comprises instructions for, when executed, performing acts of: presenting to a user an interface for receiving a list of experimental factors, one or more levels for each experimental factor, and one or more times at which one or more experimental factor values are to be set and/or one or more measurements are to be taken; receiving, as input, the list of experimental factors and the one or more times; creating a data structure corresponding to the received one or more experimental protocols, using the received factorial design, each experimental protocol comprising one or more values for the experimental factors and one or more times; and assigning each experimental protocol to a specific cell culture contained in a reactor array.

The program, according to some embodiments, comprises instructions for, when executed, performing acts of: receiving user input defining a plurality of reactor arrays, each comprising a plurality of reactors; receiving user input defining at least one reaction factor that independently is selectable to operates on each reactor; receiving user input defining at least one array factor that simultaneously, but not independently, operates on each reactor within a reactor array; receiving user input for each reaction factor and each array factor, defining a plurality of levels; and generating a plurality of experimental protocols using, for each reaction factor and each array factor, a respective level selected from the plurality of levels, where at least one protocol includes at least one reactor array that comprises a first reactor operated on by a first level of a reaction factor, and a second reactor operated on by a second level of the reaction factor.

In yet another aspect, the program comprises instructions for, when executed, performing acts of providing a plurality of reactor arrays, each comprising a plurality of reactors; defining at least one reaction factor that may, if selected in an experimental protocol, independently operate on a selected reactor; defining at least one array factor that simultaneously, but not independently, operates on each reactor within a single reactor array; for each reaction factor and each array factor, defining a plurality of levels; and generating one or more sets of experimental protocols applicable to the plurality of reactor arrays such that at least one set of the one or more sets includes (1) a first experimental protocol applicable to a first reactor of a particular reactor array selected from the plurality of reactor arrays, and (2) a second experimental protocol applicable to a second reactor of the particular reactor array. The first experimental protocol and the second experimental protocol may each be generated using, for each selected reaction factor and each selected array factor, a respective level selected from the plurality of levels, where the first experimental protocol includes a first level of a reaction factor, and the second experimental protocol includes a second level of a reaction factor that is different from the first level. In certain cases, the method also includes an act of applying the set of experimental protocols to the corresponding particular reactor array using an automated device.

In still another aspect, the program comprises instructions for, when executed, performing acts of: receiving user input defining a plurality of elements and a plurality of groups containing the plurality of elements, at least one group containing more than one element; receiving user input defining at least one elemental factor and at least one group factor, where each elemental factor independently operates on each element, and each group factor simultaneously, but not independently, operates on each element within a group; for each elemental factor and each group factor, defining a plurality of levels; and generating a plurality of protocols to operate on each group and each element within each group, using, for each elemental factor and each group factor, a respective level selected from the plurality of levels, where at least one protocol includes at least one group containing at least a first element and a second element where the first element is operated on by a first level of an elemental factor, and the second element is operated on by a second level of the elemental factor.

In another aspect, the present invention is directed to a method of making one or more of the embodiments described herein. In yet another aspect, the present invention is directed to a method of using one or more of the embodiments described herein. In still another aspect, the present invention is directed to a method of promoting one or more of the embodiments described herein.

In yet another aspect, a method of screening cell culture experiments comprises collecting in a data store a plurality of descriptors of automated cell culture experiments which have been performed, and resulting experimental data; and operating a computer to search the data store for descriptors matching provided descriptor criteria. Collecting descriptors and experimental data may include storing said descriptors and experimental data separately. Collecting descriptors may include aggregating from multiple sources descriptors of experiments conducted by those sources. Operating a computer to search may include operating a computer to search only descriptors for which the searcher or search requester (interchangeably, a “searcher”) is authorized. A fee may be charged for searching the data store, for adding a descriptor to the store, for adding experimental data to the store, or for retrieving information associated with a search result.

Another aspect is computer-readable medium having recorded thereon signals defining operations for performing the method and constituting the apparatus of any of the foregoing methods and apparatuses, when executed on a processor.

Still another aspect it a computer-readable medium having recorded thereon a descriptor usable for defining an experiment to an automated cell culture device and encoding desired experimental conditions for a cell culture experiment.

A further aspect is a method of performing cell culture experiments comprising recording as a descriptor in a machine-readable form the parameters and specifications for performing an experiment, in a format usable, directly or indirectly, by an automated cell culture device. The method may further include recording measurements from said experiment in a machine-readable form which associates the measurements with a corresponding descriptor for the experiment. An interpreter program, or parser, may be used to interpret a set of one or more related descriptors as a set of desired experimental conditions and generate a corresponding sequence of commands to direct an automated system to execute corresponding experiments in bioreactors manipulated by the automated system. Such an interpreter or parser will be specific to the particulars of the descriptor format and the command set and syntax of the cell culture apparatus, and its writing or design is within the average skill of software engineers. The bioreactors may be arranged in arrays and the method may further include mapping experimental parameters to the arrays to minimize the number of arrays required by an experiment.

Another aspect is a method of performing a factorial or multi-factorial experimental design including operating a computer to assist a user in the creation of a set of machine-readable descriptors corresponding to said factorial design. Such method may further include using the descriptors as “tags” to index data resulting from the experiment for later search and analysis.

Still another aspect is a method of facilitating efficient cell culture experimenting comprising searching previous cell culture experimental results recorded in a data store by comparing tags of data sets in said store and returning results ranked by degree of similarity to the query tag.

Such aspects may appear alone or in any non-conflicting combination, in particular embodiments.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 shows a top view of a system for controlling a chemical, biological, and/or biochemical sample, according to one embodiment of the invention;

FIG. 2 shows a top view of a system according to another embodiment of the invention, including a plurality of handling devices;

FIGS. 3A-3B are flowcharts illustrating the use of an embodiment of the invention;

FIGS. 4A-4D illustrate an example of a constrained factorial design, according to another embodiment of the invention;

FIGS. 5A-5C illustrate examples of descriptors, according to certain other embodiments of the invention;

FIG. 6A is an illustration of an example of part of a user interface screen as might be used to allow the user to enter a description of an experiment;

FIG. 6B is an illustration of an example of part of a user interface screen as might be used to allow the user to select options such as configuring the physical resources required for an experiment;

FIG. 7 is an illustration of an example of part of a user interface screen as might be used to allow the user to input at least some system configuration information;

FIG. 8 is an illustration of an example of part of a user interface screen as might be used to allow the user to input incubator control data;

FIG. 9 is an illustration of an example of part of a user interface screen as might be used to allow the user to input measurement information;

FIG. 10 is an illustration of an example of part of a user interface screen as might be used to allow the user to input scheduled measurement parameters;

FIG. 11 is an illustration of an example of part of a user interface screen as might be used to allow the user to input parameters for scheduled sampling operations; and

FIG. 12 is an illustration of an example of part of a possible *.ini-style descriptor file for an experiment.

### DETAILED DESCRIPTION

There is presented herein systems and methods which relate to the computer-facilitated design of large-scale, multi-factorial cell culture experiments and the like, and to the control of reaction sites and/or arrays of reaction sites to perform such experiments using automated devices (often called robots”). In certain cases, the invention is directed to controlling a plurality of cell culture experiments, e.g., using an automated cell culture device. In one set of embodiments, a data structure or a “descriptor” is provided for use with cell culture experiments. The descriptor may be maintained within a data store and may be used, for instance, to control one or more cell culture experiments, to identify one or more cell culture experiments, and/or to identify or “tag” data arising from one or more cell culture experiments, e.g., for further analysis or recall. Another set of embodiments is generally directed to generating experimental protocols (e.g., within an experimental framework) for controlling reaction sites and/or arrays of reaction sites (e.g., containing one or more cell cultures), for example, using a factorial design or a “constrained” factorial design, where not all of the factors can operate independently. For example, instead of experimenting one at a time on individual cell cultures, multiple reaction sites may be arranged in an array (e.g., in a chip or other physical article), and the array of reaction sites may then be manipulated with some factors, such as temperature, operating on the entire array of reaction sites, rather than independently on one reaction site. The constrained factorial design is not limited to only cell cultures or reaction site arrays, but can also be used in any factorial design where a plurality of elements, and a plurality of groups containing the plurality of elements, are present, where one or more factors simultaneously, but not independently, operate on each element within the group. The descriptor may be employed as input to a parser which, in turn, generates corresponding commands or signals to control the automated device, to conduct the experiments. Other embodiments of the present invention are directed to machine- and/or computer-readable media implementing any of the above, e.g., for use in formulating an experimental design and for controlling an automated cell culture device, as well as methods of making and using such media.

The use of a data store containing descriptors of a like structure provides a basis for researchers to search for experiments to determine whether any such experiment has previously been performed. Then, depending on the way the authorization rights to the data store are managed, the querying researcher may be given access to the results of prior experiments or locked out. A querying researcher may, for example, be given only the contact information for the researcher who performed the previous experiment, so as to permit the inquirer to approach the other party to work out terms for access to the data. Authorizations for access may be mediated by a third party or the researchers' institutions may establish standing access agreements, to name just a couple of variants that are foreseeable. The data store may be owned and maintained by an entity that charges for access to its contents, or access may be free, depending on business decisions. As an adjunct to use of such systems, an entity may operate a service, searching upon request one or more data stores of descriptors and providing search results. The results could range from a simple yes/no statement as to a match being found, to sharing actual experimental results on some agreed basis. The data store may contain descriptors and data of multiple parties' experiments and could be a central service bureau for the public or subscribers or entities having some other relationship.

Various aspects of this invention generally relate to controlling chemical, biological, and/or biochemical samples positioned within a reaction site, for example, contained within a chip that includes one or more reaction sites. The reaction sites within the chip may be constructed and arranged to allow a physical, chemical, biochemical, and/or biological reaction to occur therein during use of the chip. In one aspect, the invention relates to controlling chips in one or more modules addressable by one or more handling devices, for example, positioned so as to surround the handling device. In some cases, many chips may be controlled to perform experiments (or otherwise act) on dozens or even hundreds or more reaction sites and/or chips. These reactors may be controlled, e.g., sequentially or in parallel, for example, through the use of robotics, for example, which can control the chips automatically, for instance, to move them between modules and/or effectuate experimental or manufacturing process design (e.g., per appropriate descriptors). Certain embodiments of the invention may be used, for example, to promote or optimize chemical, biological, and/or biochemical synthesis and/or cell or biological culture or growth, for instance, for the production of compounds such as drugs, proteins, and/or other therapeutics, experimentally or commercially. Thus, embodiments of the invention may be used to design and to execute, under computer control, a plurality of reactors, e.g., hundreds or thousands of reactors, with minimal user action required. A considerable savings in time results and even after considering equipment cost, the cost per experiment may be much reduced.

A “microbioreactor array,” or a “chip,” as used herein, is an article that includes one or more reaction sites. Typically, the chip is a generally flat or planar article (i.e., having one dimension that is relatively small compared to the other dimensions); however, in some cases, the chip is non-planar. The reaction sites may be arrayed thereon in any suitable configuration, e.g., linearly, in a matrix or rectilinear configuration, etc., depending on the shape of the chip. The chip can be fabricated using any suitable manufacturing technique or combination of techniques. Non-limiting examples of potentially suitable fabrication processes include wet etching, chemical vapor deposition, deep reactive ion etching, anodic bonding, injection molding, hot pressing, and/or LIGA. For example, the chip may be fabricated, at least in part, by etching or molding silicon or other substrates, for instance, via standard lithographic techniques. The chip may also be fabricated using microassembly or micromachining methods, for example, stereolithography, laser chemical three-dimensional writing methods, modular assembly methods, replica molding techniques, injection molding techniques, milling techniques, and the like as are known by those of ordinary skill in the art. Examples of materials that can be used to form chips include polymers, silicones, glasses, metals, ceramics, inorganic materials, and/or any combination of these or other materials.

Non-limiting examples of chips potentially suitable for use with the present invention include those disclosed in U.S. patent application Ser. No. 10/119,917, filed Apr. 10, 2002, entitled “Microfermentor Device and Cell Based Screening Method,” by Zarur, et al., published as 2003/0077817 on Apr. 24, 2003; U.S. patent application Ser. No. 10/633,448, filed Aug. 1, 2003, entitled “Microreactor,” by Jury, et al., published as 2004/0121454 on Jun. 24, 2004; U.S. patent application Ser. No. 10/457,049, filed Jun. 5, 2003, entitled “Materials and Reactor Systems having Humidity and Gas Control,” by Rodgers, et al., published as 2004/0058437 on Mar. 25, 2004; U.S. patent application Ser. No. 10/457,015, filed Jun. 5, 2003, entitled “Reactor Systems Having a Light-Interacting Component,” by Miller, et al., published as 2004/0058407 on Mar. 25, 2004; U.S. patent application Ser. No. 10/664,046, filed Sep. 16, 2003, entitled “Determination and/or Control of Reactor Environmental Conditions,” by Miller, et al., published as 2004/0132166 on Jul. 8, 2004; U.S. patent application Ser. No. 10/664,068, filed Sep. 16, 2003, entitled “Systems and Methods for Control of pH and Other Reactor Environmental Conditions,” by Miller, et al., published as 2005/0026134 on Feb. 3, 2005; U.S. patent application Ser. No. 10/664,067, filed Sep. 16, 2003, entitled “Microreactor Architecture and Methods,” by Rodgers, et al., published as 2005/0032204 on Feb. 10, 2005; U.S. Provisional Patent Application Ser. No. 60/577,986, filed Jun. 7, 2004, entitled “Reactor Mixing,” by Johnson, et al.; U.S. Provisional Patent Application Ser. No. 60/577,977, filed Jun. 7, 2004, entitled “Gas Control in a Reactor,” by Rodgers, et al.; U.S. Provisional Patent Application Ser. No. 60/609,721, filed Sep. 14, 2004, entitled “Inlet Channel Volume in a Reactor,” by Miller, et al.; or U.S. Provisional Patent Application Ser. No. 60/636,420, filed Dec. 14, 2004, entitled “Creation of Shear in a Reactor,” by Johnson, et al., each incorporated herein by reference.

A reaction site is a site defined within a chip that is constructed and arranged to produce a physical, chemical, biochemical, and/or biological reaction during use of the reaction site. More than one reaction site may be present within a chip in some cases. In certain embodiments, the reaction site may also include one or more biologicals, for example, cells and/or tissues.

The volume of the reaction site can be very small in some embodiments. Specifically, for many cell culture reactions, the reaction site may have a volume of less than one liter, less than about 100 ml, less than about 10 ml, less than about 5 ml, less than about 2 ml, less than about 1 ml, less than about 300 microliters, less than about 100 microliters, less than about 30 microliters, or even less than about 10 microliters in various embodiments. The reaction site may also have a volume of less than about 5 microliters, or less than about 1 microliter in certain cases. The reaction site may have any convenient size and/or shape. If more than one reaction site is present within a chip, each reaction site may independently have the same and/or different sizes and/or shapes.

In one set of embodiments, a system of the invention may comprise a cluster tool-type device adapted to control chemicals, biochemicals, and/or biologicals including, but not limited to, cells and/or tissues. A “cluster tool,” as used herein, is a device that can move objects between different locations, typically “modules,” where the objects are stored and/or subject to different testing and/or treatment conditions. Cluster tools may include one or more automated actuators (e.g., a handling device) that can rotate about a vertical axis, generally surrounded (e.g., radially) by modules into which and from which objects can be introduced and removed for various testing and/or treatment steps. In some cases, the handling device may be an articulated arm, e.g. having a mechanical claw for releasable holding one or more chips. As used herein, an “automated” system or device refers to a system or device that is able to function without human direction, for example, as further described below. That is, an automated system can perform a function during a period of time after a human has finished taking any action to promote the function, e.g. by entering instructions into a computer. Typically, automated systems can perform repetitive functions after this point in time. Sensors, control systems, or the like may also be positioned to facilitate control of the system. One non-limiting example of such a system is disclosed in U.S. patent application Ser. No. 10/863,585, filed Jun. 7, 2004, entitled “System and Method for Process Automation,” by Rodgers, et al., published as U.S. Patent Application Publication No. 2005-0037485 on Feb. 17, 2005, incorporated herein by reference.

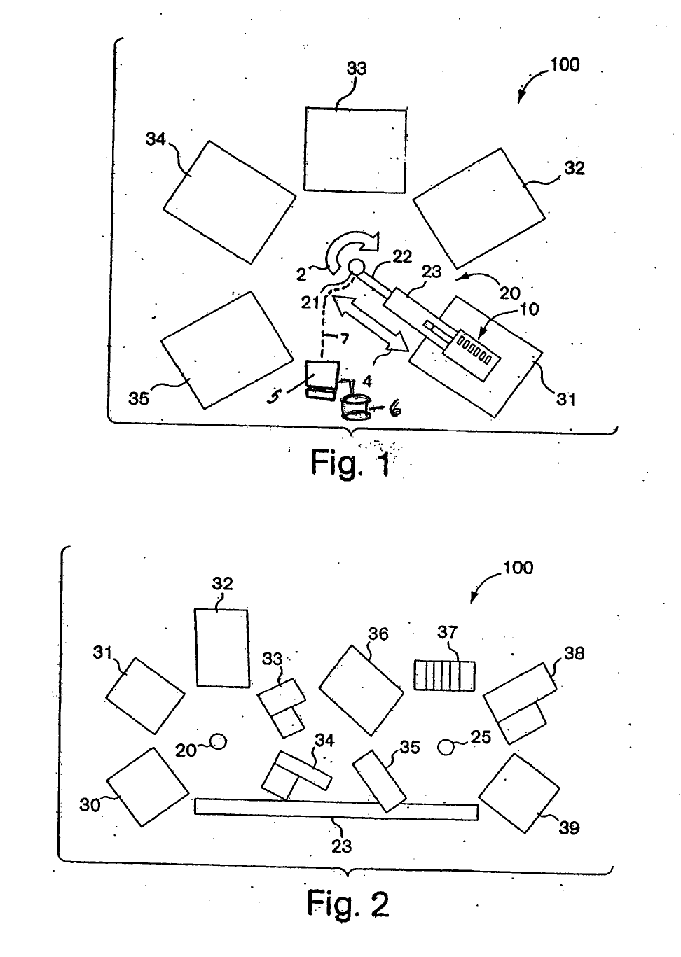

Referring now to the figures, in FIG. 1 system 100 (shown diagrammatically in a top view) includes handling device 20, and a plurality of modules 31, 32, 33, 34, 35 positioned so as to be addressable by the handling device (generally surrounding the handling device in the embodiment illustrated). Handling device 20 may be automated and/or under manual control. In FIG. 1, handling device 20 includes a central pivoting mechanism 21 that pivots on a vertical (into the plane of the paper) axis, an arm 22 emanating from the central pivoting mechanism for addressing the various modules, and a sample securing mechanism 23 constructed and arranged to secure a sample (e.g., a chip) and to introduce and/or remove the sample from at least one, and preferably all of the modules addressable by handling device 20. Securing mechanism 23 can be a clamp, a detent mechanism, a mechanism including protrusions insertable into corresponding indentations in a chip, or the like. As shown, a chip 10 is secured by securing mechanism 23, and handling device 20 is able to move chip 10 about system 100. Pivoting mechanism 21 is able to rotate chip 10 about an axis perpendicular to the plane of the paper (indicated by arrow 2, with the axis aligned with the center of mechanism 21), while arm 22 is able to move chip 10 in a direction substantially perpendicular to the axis (i.e., in a radial direction towards or away from the axis, as indicated by arrow 4) and/or substantially parallel to the axis (i.e., in a direction perpendicular to the plane of the paper, direction not shown).

Radially positioned around handling device 20 are a series of modules 31, 32, 33, 34, 35. The modules are arranged such that handling device 20 is able to add or remove a chip to or from any of the modules. It should be noted that, although FIG. 1 illustrates a rotational apparatus able to, independently, rotate a chip about an axis, and translationally move the chip in at least one of a direction substantially perpendicular to the axis and a direction substantially parallel to the axis, that in other embodiments, other devices may be used to move a chip from one module to another. As a non-limiting example, the handling device may include a multi-axis articulate robot having one or more arms sufficiently articulated so as to be able to retrieve and/or position a chip within a module. For instance, the handling device can include an “arm” having one or more articulated joints (for example, a shoulder, an elbow, and/or a wrist joint). As additional, non-limiting examples, the handling device may include a cylindrical apparatus, a linear translation stage, an elevator mechanism, a conveyor belt, etc.

The handling device may secure and/or transport one or more chips to and from one or more modules located proximate the handling device (e.g., per an experimental descriptor, as further discussed below). The handling device may control the chips, for example, in response to a user or in an automated sequence. For instance, in FIG. 1, handling device 20 can position a chip in a first module (which may be any module accessible to handling device 20), allow the module to perform a manipulation on the chip (for example, testing and/or treatment, as described below) then move the chip from the first module to a second module. In one embodiment, the handling device may include one or more effector mechanisms able to secure or “grab” a chip from a module, and/or position a chip within a module. Those of ordinary skill in the art will be able to chose appropriate mechanisms able to secure and/or position chips.

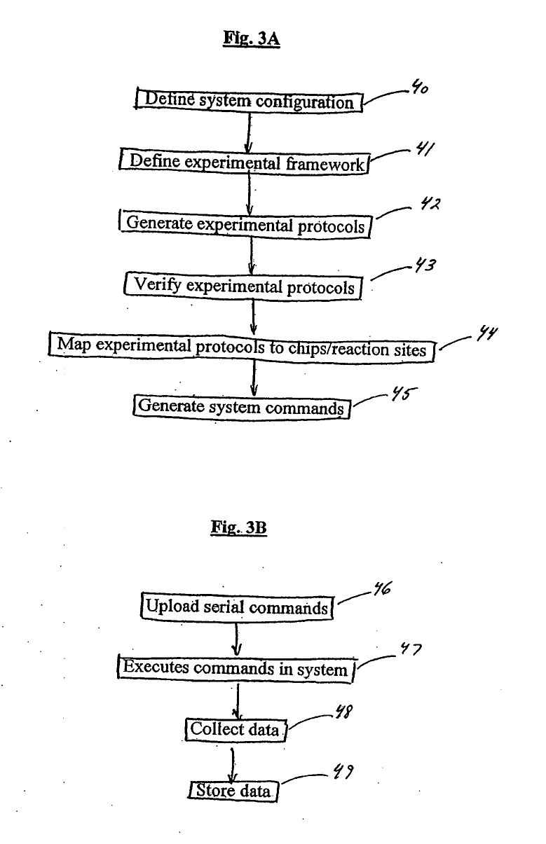

In some embodiments, the system may include more than one handling device, for example, as illustrated in FIG. 2. In this figure, system 100 includes two handling devices 20, 25, and a series of modules disposed around the two handling devices. Modules 30, 31, 32, 33, and 34 are arranged to be accessible to handling device 20, while modules 35, 36, 37, 38 and 39 are arranged to be accessible to handling device 25. Conveyor system 23 can be used to transport a chip between handling device 20 and handling device 25.

The handling device may move chips between modules in response to, for example, an experimental protocol as further described herein, instructions from a user, sensor measurements, etc. As used herein, a “module” is an apparatus able to contain and/or perform a manipulation on a chip. For example, the module may hold a chip (e.g., for a finite period of time or under certain environmental or other conditions), heat and/or cool the chip, determine the identity of a chip (or a component or substance therein), perform a measurement on the chip, add or remove a substance from the chip, perform an assay on the chip, control the pH of the chip, allow a reaction and/or an interaction to occur within the chip, measure the concentration of one or more species within the chip (such as oxygen, carbon dioxide, nitrogen, reagents, cells, media, or nutrients, for example, glucose, glutamine, pyruvate, and/or various amino acids, vitamins, hormones, serum, ions, etc.), and/or determine an analyte within the chip, for instance as in a product titer, a protein titer, an antibody titer, a cell titer, a hormone titer, a small molecule (i.e., a molecule having a molecular weight of less than about 1000 Da) titer, a peptide titer, a ligand titer, etc. As another example, if a chip contains one or more cells, a module may determine one or more characteristics of the cells, for example, cell concentration, cell density, cell viability, cell yield (e.g., of a product), cell productivity, cell type, cell morphology, cell adhesion, etc. Any of the above modules within the system can be replaced or substituted as desired, for example, to suit the needs of a particular application. In some cases, the modules are designed to be interchangeable. The modules may be replaced between operation cycles of the system, and/or even while the system is being operated. In certain embodiments, one or more modules and/or handling devices may be enclosed within a housing, for example, to maintain cleanliness and/or sterility of the interior of the modules and/or any chips contained therein.

Examples of modules that can be used with the invention include, but are not limited to: “stack” or “holding” module that can store or contain chips, optionally in a sterile environment; a sterilization module able to sterilize a chip (for example, through raising the temperature or the application of ionizing radiation); an identification module that can detect or determine specific chips (for example, using identifying characteristics such as colors or bar codes, radio-frequency tags, or memory or other semiconductor chips); a data transfer module able to read or write data to or from a chip; a fluid transfer module able to add and/or remove a substance to a chip (e.g., a fluid, or a substance contained within a fluid), for example reagents, chemicals, cells, media, pH buffers, initiators, etc.; a sensor module able to determine and/or record an condition within the chip, such as an environmental condition, for example, pH, temperature, atmospheric conditions (e.g., gas concentrations), humidity, dissolved oxygen or carbon dioxide concentration, the concentration of nutrients or other chemicals within the chip (e.g., within the media), cell density, cell viability, cell morphology or other cell characteristics; an imaging module able to acquire an image of a chip or a portion thereof, such as a reaction site (e.g., optically, fluorescently, etc.); a refrigeration module; an incubation module able to maintain the temperature and/or other atmospheric conditions (such as the relative humidity) at a predetermined level, for instance, to provide a desired environmental condition or a range of environmental conditions; a sampling module able to remove a substance from a chip (e.g., media, cells, products, etc.); an assay module able to perform chemical or biological assays on a chip; an actuating module for physically manipulating (e.g., agitating) a chip; or the like. Combinations of these and/or other modules are also envisioned, for example, a module that can fill and incubate a chip. Further examples of these and other module functions are further described below.

As an example of a module function, in some embodiments, at least one of the modules is able to hold or contain a chip for a certain length of time (e.g., a “holding” module, or a “stacking” module), for example, while the handling device is manipulating other chips, or where a certain amount of time is necessary before the chip can be moved to the next step and/or the next module. For instance, the holding module may be used for aging samples and/or storing samples between other activities involving other modules.

As another example of a module function, a module may be able to identify one or more chips contained therein. In one embodiment, the module has an identification system able to read an identification tag associated with a chip, such as a bar code, a serial number, a color tag, a radio tag, a magnetic tag, a radio-frequency tags, or memory or other semiconductor chips, or another identifying characteristic.

As yet another example of a module function, a module may be a data transfer module able to read and/or write data to the chip. For example, the data read and/or written to the chip may include identification data, operating or environmental condition data, results of assays or other manipulations to the chip, etc.

In another example of a function of a module, in some cases, a module may be able to determine and/or control the internal environment within the module (e.g., a “sensing” module), and/or within a chip (or a portion thereof, such as within a reaction site). Determination and/or control of the environment within the module and/or within the chip may be achieved, for example, using one or more sensors, processors, and/or actuators positioned on and/or in and/or proximate the module.

In yet another example of a function of a module, a module may be able to add (e.g., a “filling” module”) or remove (e.g., a “sampling” module) a species to a chip, or a component within the chip, such as a reaction site. In some cases, the module may be able to both add and remove a species to a chip. For example, the module may include a fluid transfer system able to add and/or remove a fluid, or a substance contained within a fluid to and/or from the chip or a component thereof, such as to a reaction site, e.g., in response to an actuator and/or a sensor. For example, the fluid transfer system may add and/or remove a specified amount of chemicals, initiators, raw materials, liquids, pH buffers, cells, media, reagents, products, etc. As another example, the fluid transfer system may remove a sample from the chip, for example, for analysis, or for further processing.

In still another example of a function of a module, a module may include a sterilization system able to sterilize a chip, for instance to kill or otherwise deactivate biological cells (e.g., bacteria), viruses, etc. therein. The sterilization system may sterilize the chip using chemicals, radiation (for example, with ultraviolet light and/or ionizing radiation), heat-treatment (e.g., raising the temperature above the boiling point of water), or the like. Appropriate sterilization techniques are known to those of ordinary skill in the art.

In yet another example of a function of a module, a module may include a positioning system able to position a chip within the module. Those of ordinary skill in the art will know of suitable positioning systems for use within a module.

Combinations of the above functions and/or other functions may be included within a module. Thus, in one embodiment, the module is configured to be able to perform an assay on a chip, or a component within the chip, such as a reaction site, for example, using a combination of sensors, processors, control system, etc. For example, the module may be configured to perform a biological assay on the chip (or on components within the chip), such as an ELISA, an immunoassay, an affinity binding assay, a blotting assay, a spectrometric determination, a polarization determination, or the like. For instance, the module may be configured to be able to perform a biological, chemical, and/or biochemical assay automatically, in conjunction with monitoring or sensing of the chip by a sensor. Those of ordinary skill in the art will readily envision other assays that can be adopted for use with the invention.

Other non-limiting examples of modules that may be provided in certain embodiments to manipulate a chip include certain commercially available devices, for example, Freedom EVO, Genesis RSP, or Genesis NPS, each from Tecan (Maennedorf, Switzerland). In certain embodiments, one or more modules and/or handling devices described above may be controlled by an operator (e.g., a mechanical or automated system, or a human user). A system according to certain embodiments may be configured so that a human user may control operation of the modules and/or handling devices (manually or automatically), for example, using a user interface (such as a control panel) or a computer, as further described herein. In other embodiments, however, a system may be programmable and/or automated, for example, such that the system is able to automatically respond to certain conditions, or reach a certain level of productivity. Of course, in some embodiments, a system can be both user-controlled and automated: for example, in cases where a user is able to override or alter an automated program.

In some embodiments, the system includes a user interface (provided, for example, by a suitably configured or programmed computer or just a control panel, possibly with a video display screen 12 and one or more input devices such as a keyboard and mouse) that may be configured to allow a user to design and/or control and/or monitor any aspect of an experiment or series of experiments being performed by the system. For example, the user interface may be configured to allow automated or manual control of any or all of the modules and/or handling devices. The user interface may also allow the analysis, determination, storage, logging, searching, handling, tagging, etc. of data generated by the system, and in some cases, on an automated or at least partially automated basis. In some cases, the data may be determined and/or analyzed in real-time, e.g., while an experiment is being performed by the system. The same computer may be used to provide a user interface for “building” an experiment and for controlling and/or monitoring experiment execution, or different computers and/or interfaces may be used. Indeed, a computer/interface used to design an experiment may be co-located with the system 100 or it may be remote therefrom, with or without a communication link between them. A non-limiting example is shown in FIG. 1. In this figure, a computer 5, provides a user interface, or UI (e.g., a computer program which generates a UI), which can be used to control and/or monitor the system. Optionally, as further discussed herein, the computer may allow a user to design experiments—i.e., generate one or more experimental protocols to be performed by the system. Commands may be sent from computer 5, through link 7 (e.g., a cable, a system bus, a wireless interface, etc.), to handling device 20. The computer may be programmed in any suitable language, for example, but not limited to, Java, Perl, C, C#, or C++, FORTRAN, Pascal, Eiffel, Basic, COBOL, machine language, etc., or any of a variety of combinations thereof.

In some cases, the system may include, at computer 5 or at another computer located at the same or some other location and appropriately interconnected, a data management system (e.g., appropriate computer programming and a data store(s) 6). The data management system may be configured to allow, for example, searching of data generated by the system. Such data includes experimental design data as well as experimental measurements. In some cases, a descriptor may be used to facilitate tagging, searching, and/or storage of the data, e.g., as further described below. That is, an experiment may be recorded in a structured way, in a file(s) or record(s), so as to facilitate a search to determine prior experiments and results. The data generated by the system may include, but is not limited to, the initial state of one or more chips, concentrations of one or more substances (e.g., reagents, nutrients, etc.) over time, the type of cell line (if any), cell density over time, type of media, the pH, temperatures or pressures within the system or in chips within the system over time, the set points of pH or other controlled conditions, environmental or other conditions (such as atmospheric conditions) within the system or in chips within the system, identification of chips within the system (e.g., using a bar code, etc.), data acquired from sensors or assay modules, images such as optical or fluorescent images, time data (e.g., time stamps), etc. The data may also be exported to other platforms for further analysis in some cases. In certain cases, data from multiple reaction sites and/or chips may also be compared. Preferably, the generation of a descriptor for an experiment, in an analyzable, searchable, uniform format, both associates the data with the experimental process that generated it (i.e., a protocol) and allows searching of protocols and examination of results of past experiments.

The user interface, in certain embodiments, may be configured to allow a large number or lists of factors to be analyzed, for example, as in factorial design. The user interface may be configured so that factors leading to an optimized solution (e.g., maximizing a reaction rate, chemical yield, enantioselectivity, or, in the case of cells, cell growth, cell yield, cell division, production of one or more desired compounds, etc.) may be chosen for further study, and/or for further scale-up and/or “numbering-up.” As an example, an experiment and/or series of experiments may be performed where each of several (even tens, hundreds or even thousands of factors) are varied systematically or randomly, e.g., using factorial design or a constrained factorial design over several levels, with conditions or experimental factors changing at programmed times, as discussed in more detail below. There may be, therefore, hundreds or thousands of combinations of conditions tested.

As a non-limiting example, where cells are present in a system, experimental factors that can be determined include, but are not limited to, temperatures, pressures, initial pHs, pHs during a reaction, media compositions (e.g., glutamine, sugars, carbohydrates, hormones, vitamins, serum, sources of nitrogen and/or carbon, etc.), flow rates, dissolved gas concentrations (e.g., O₂, CO₂, N₂, etc.), cell types, cell densities, cell cycle positions, cell dimensions, substrates, shear rates, gas concentrations, relative humidities, cell synthesis or production rates, cell replication rates, etc. Optimized conditions could be selected, e.g., for further study, or for scaling or “numbering up.” In certain embodiments, it is possible to simultaneously process more than one chip, as described herein. In some cases, the number of arrays provided may be selected so as to produce a certain quantity of a species or product, or so as to be able to process a certain amount of reactant at a certain rate. Thus, certain embodiments of the invention are amenable to scalability and parallelization.

In some aspects, the invention allows a user to design multiple experiments (for example, within an experimental framework), which may be automated, and optionally, program a system to perform such experiments—for example, a system including a cluster tool-type device, e.g., as previously described. For instance, a user can designate certain experimental factors which are to be varied (for example, including multiple levels), and then create one or more experimental protocols (e.g., using a computer program) that can be performed by the device, in which one or more of the factors are altered. For example, in cell culture and other similar chemical, biochemical, and/or biological reactions, the experimental protocols for the cell culture or other reaction may need to be optimized, i.e., with respect to temperature, pressure, concentration of a reagent, concentration of a product, agitation or mixing conditions, cell nutrients, cell density, protein production, protein extraction, or the like. In many cases, the optimization process may require multiple experiments to be performed to assess the effect of the various factors on the optimization of the reaction. The present invention thus provides, in some aspects, systems and methods for automatically preparing protocols for such experiments, and in some cases, for conducting such experiments as well.

Thus, in one aspect, a user inputs, into a computer, an experimental framework, selecting experimental factors (e.g., from a list) that are desired to be varied (e.g., temperature, pressure, etc.), including multiple levels or values for each factor (e.g., for temperature, 36° C., 37° C., 38° C., etc.). It should be noted that the levels can be continuous (e.g., temperature, concentration, etc.), or discrete (e.g., the presence/absence of a catalyst, cell type, etc.), depending on the type of factor. The computer then generates one or more experimental protocols based on these inputs, each of which uses one or more levels chosen for each factor to be varied. The factors may be varied by the computer, for example, independently of each other, or on a dependent basis. Of course, if so desired, one or more experimental protocols may be replicated (for example, each experimental protocol may be performed twice, three times, four times, five times, etc.).

After the experimental protocols have been formulated, the experimental protocols may be performed, either manually, or automatically, for instance, using an automated system, e.g., a system including a cluster tool-type device and/or an automated cell culture device as described above. The computer may perform a “mapping” operation, in which one or more experimental protocols are mapped to one or more reaction sites in one or more chips. In some cases, the mapping may be performed to ensure that each chip (which may contain more than one reaction site, and more than one experimental protocol mapped to the chip) will not be subjected to conflicting or inconsistent conditions. For example, a constrained factorial design may be used, as further discussed herein.

Before proceeding, the computer may also verify that the experimental protocols can be adequately performed. For instance, the experimental protocols may require equipment that is not currently available. As an example, an experimental protocol may require 5 pumps, yet the system may have only 4 pumps available. As another example, an experimental protocol may be generated to require a flowrate that is higher than the maximum flowrate that a pump can produce. The computer may also verify that adequate supplies are available to perform the experimental protocols, for example, the amount of reagent, the number of cells, etc. The computer may also determine (and report) the time necessary to perform the experimental protocols (for example, using one or more Gantt charts). If the computer determines that a certain condition of the experimental protocol is impossible or at least impractical, e.g. due to conflicting constraints or lack of resources, the computer can then alert the user, who can alter the experimental protocols and/or the system as necessary.

The computer may then prepare or convert the experimental protocols into a series of commands to be sent to the automated system. For example, a series of commands may be sent to a handling device in a cluster tool-type device, as previously described. When executed by the handling device, the system may thus perform the experiment specified by the experimental protocols.

To minimize the number of chip required, in some cases, the computer may optimize multiple experimental protocols to be performed simultaneously and/or sequentially by the handling device, for example, multiple experiments on one chip and/or on multiple chips.

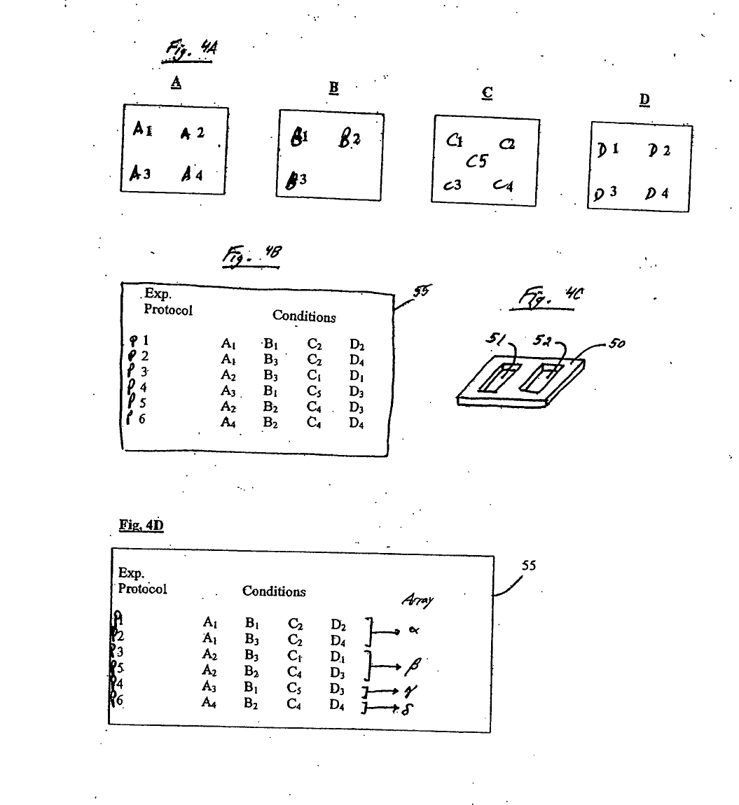

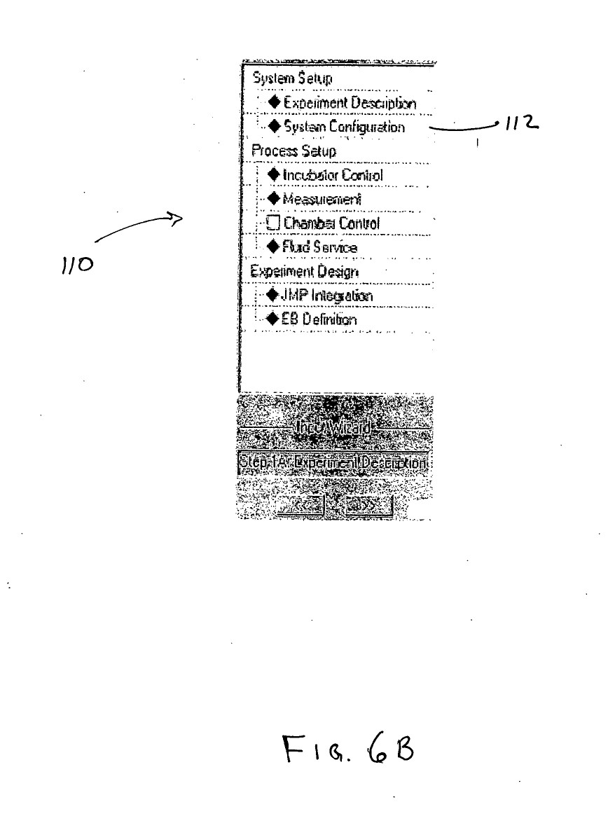

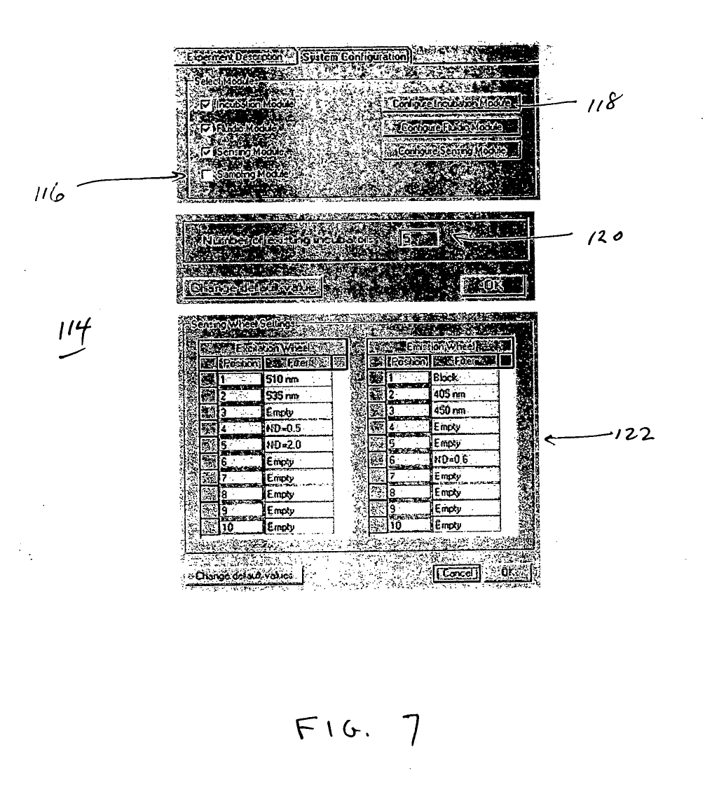

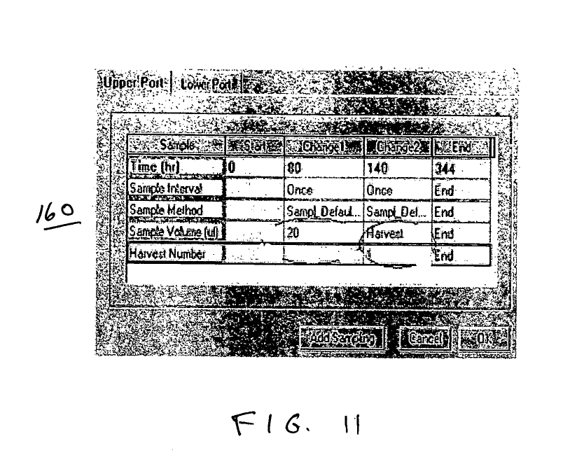

A non-limiting example flowchart of this process is shown in FIG. 3A, which should be considered in relation to FIGS. 6A-11. FIGS. 6A-11 illustrate screens or portions of screens a user may see on a typical user interface implementing aspects of the approach taught herein, and aspects of the software for an example of a system and method. As a preliminary matter, a user may be presented and asked to complete a screen, such as shown in FIG. 6A at 90, to provide an experimental description. This information will preferably become part of the descriptor for the experiment. Relevant information may include, for example, the experiment name, cell line, type of bioreactor array being used (possibly including specific array information such as an identifier(s) from a bar code(s), reactor characteristics, experimental duration, experiment type, feeding strategy and comments. Some of these parameters may be entered free-form and others may be selected from pre-populated pull-down lists. Per block 40, a system configuration (for example, defining an automated cell culture device such as shown in FIG. 1 or FIG. 2) is inputted into a computer or similar device, e.g., a control panel, to define the resources available in the automated system. (Note that this block can be performed later, but has to be performed before block 44.) The system configuration may be inputted by a user, or in some cases, the system is a “smart” system that, when properly assembled, will automatically be configured within the computer, e.g., a “plug-and-play” system. As shown in FIG. 6B, an implementation may present to a user a menu 110. One menu pick may be “System Configuration” 112. Selecting this item may call one or more UI screens to allow the user to configure the physical resources required for an experiment. For example, as shown in FIG. 7, a screen 114 may be displayed for input of system configuration information. For example, a check box may be selected for each module used (see check box 116, as an example). Attributes of modules may be set via further screens called through buttons such as button 118. The number of incubators may be set, as at 120, and sensing wheel settings may be input, as at 122; the entries refer to filter wavelengths. In general, system configuration involves defining the modules to be used, how they are configured, and how liquid sources are set up and defined (e.g., which pump supplies which fluid).

If the same system is used for multiple experimental frameworks, the system configuration may be inputted from a storage device, e.g., from a data store such as a pre-saved file stored on a hard drive, on a storage medium (e.g., a magnetic medium) from computer memory, etc.

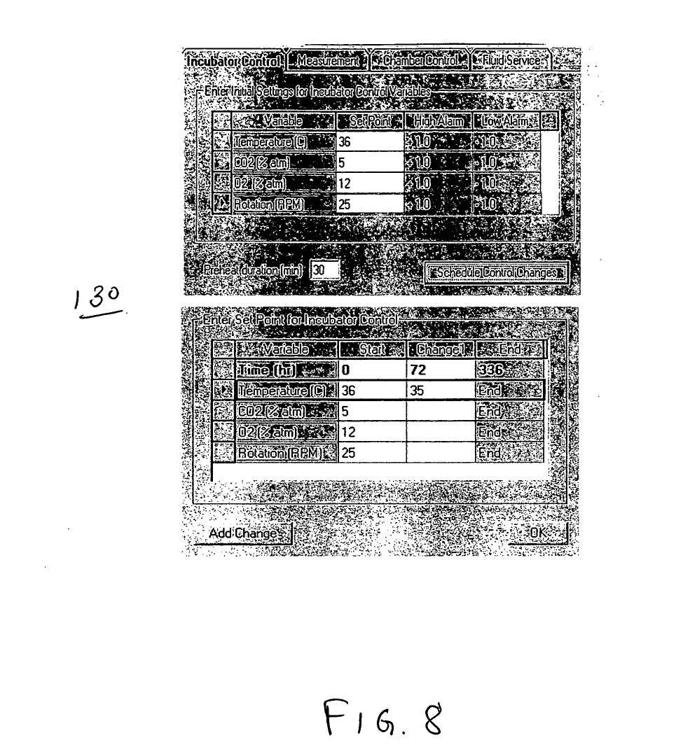

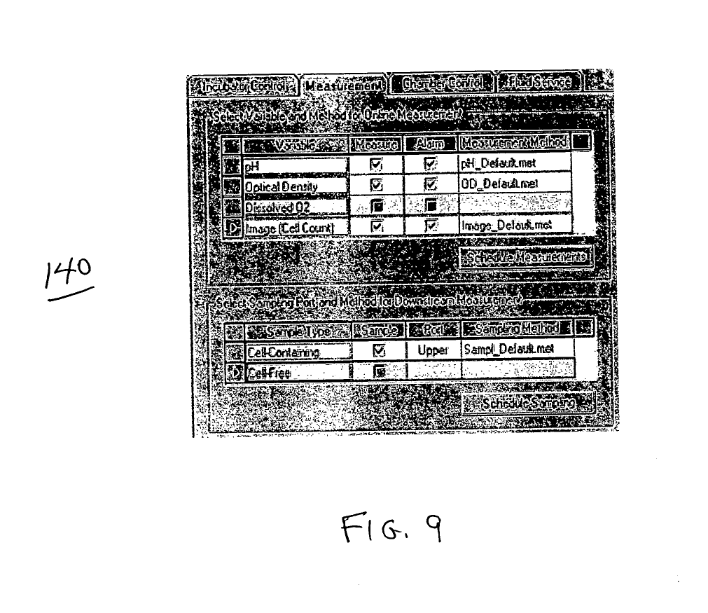

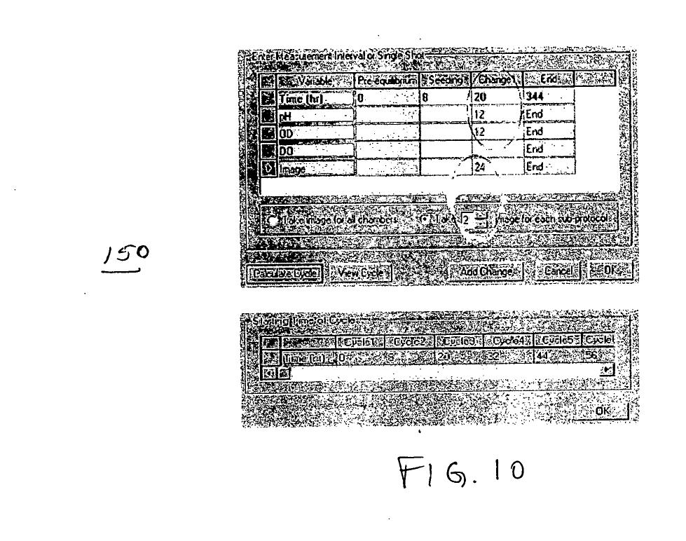

In block 41, the user inputs an experimental framework, which may include one or more factors that are desired to be varied, including multiple levels for each of those factors. A corresponding series of exemplary UI screens is presented in FIGS. 8-11. As shown in screen 130 of FIG. 8, incubator controls may be established. For example, the initial set points of environmental variables may be input, along with changes to those set points and times for making those changes. As shown in screen 140 of FIG. 9, measurements may be set up, including the variables to be measured, the type of measurement, the port through which to sample, what method to use (i.e., there may a defined library of measurement processes, with a call to one of them), how often to measure and when to change. For scheduled measurements, a page such as that shown at 150 in FIG. 10 may be completed, identifying intervals for measurements, cycles, and other factors. For scheduled sampling, a screen such as that shown at 160 in FIG. 111 may be completed. Among typical factors to be entered on such a screen are port identifications (through which a sample may be withdrawn), intervals, volume of sample to be taken (“Harvest” indicating the full chamber should be emptied), and harvest number. For chamber control, a screen such as screen 170 may be part of the UI. Among typical factors to be entered are the variables to control, any pre-existing model files for chamber setup which are to be employed, how often to change set points. Similar types of screens may be provided for setting the inputs to control one or more of chamber scheduling, fluid services (e.g., what ports to use, how much volume to dispense, how often to dispense it, and scheduled cleaning services).

In block 42, the computer then uses the user inputs (e.g., some or all of those just described, or others) to generate one or more experimental protocols, for instance, varying the levels of each experimental factor on a systematic or a random basis. In some cases, the experimental protocols may include or be associated with a descriptor, as further described herein. Optionally, but preferably, as shown in block 43, the computer may then execute code to verify that the experimental protocols can be adequately performed with the current system configuration, and if not, signal the user, allowing the system and/or the experimental framework to be altered as needed. After verification, in block 44, the computer maps each experimental protocol to one or more reaction sites and/or one or more chips. In block 45, the computer may then save the experimental protocols and/or the descriptors, e.g., for later use, and/or optionally convert the experimental protocols to a series of commands that can be sent to the (e.g., automated) system (as previously defined by the system configuration) to be executed, e.g., by a handling device. The commands may be serial in some cases, and uploaded to the handling device prior to, or during, performance of the experimental protocols by the system.

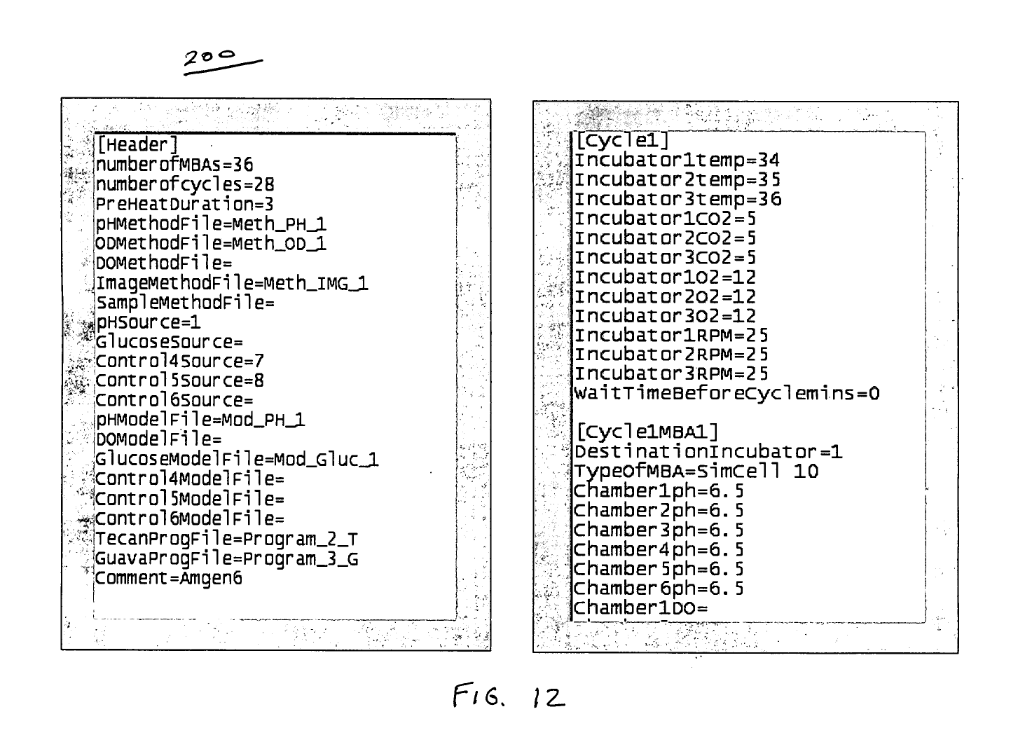

The configuration and/or experimental design data may be saved in any useful form, assuming saving to be desired. In one exemplary illustration, one or more files may be recorded in data store 6. One useful form of a file to be saved is a *.ini file, when the computer/automated device operating system is a Windows-based operating system from Microsoft Corporation. FIG. 12 shows an example, 200, of a portion of such a *.ini file. There is nothing particularly significant about this file structure. It is an example of a descriptor that can be used to encapsulate an experiment and to operate an automated device for conducting experiments.

With reference to FIG. 3B, as another example, in block 46, a series of serial commands (e.g., as produced in FIG. 3A) are sent to a handling device. The series of serial commands may be generated, e.g., using a descriptor, by the computer and/or by the handling device, in some cases. The handling device parses (i.e., interprets) and performs the commands in block 47, which may allow the system to perform one or more experimental protocols (simultaneously and/or sequentially). For instance, the handling device may manipulate one or more chips between one or more modules, which may subject the chips to different testing and/or treatment conditions. The handling device may implement more than one experimental protocol at a time, for example, one chip may have a plurality of reaction sites, where one or more reaction sites are subjected to a first experimental protocol, one or more reaction sites are subjected to a second experimental protocol, etc. In some cases, multiple chips may be processed simultaneously, each of which may be subjected to the same or different experimental protocol(s), depending on the application.

The details of parsing the commands in the descriptor file depend on the descriptor syntax and the syntax of the command language for the system 100. Those details are a matter of design implementation and those skilled in the art will readily be able to devise a parser once they know the format for the data in a descriptor and the command syntax for the system. Accordingly, such details are not part of the invention.

Data may be collected from the modules as shown in block 48, optionally tagged or identified with a descriptor (as further described herein) or otherwise associated with the experimental protocols (e.g., using a tag correlating to the descriptor, for instance, for one or more reactors) used to generate the data, and stored or “logged” in block 49 on a machine- or computer-readable medium, e.g., for later analysis, for comparison to other experimental protocols and/or other replicates of the same experimental protocols, to show regulatory compliance (for example, with FDA, ISO, and/or GMP practices, etc.), quality control purposes, or the like. For example, in one embodiment, a descriptor may be compared against an event log to verify execution of the experimental protocol, for instance, for regulatory purposes. As another example, a descriptor may be used to search experimental data and/or used to compare data between experiments (e.g., from the same or different experimental protocols). For example, the data may be searched on the basis of experimental factors such as temperature, pressure, time, reactant, product, cell type (if cells are present), run number, pH, O₂ concentration, CO₂ concentration, atmospheric conditions, relative humidity, the identity of a chip and/or a reaction site, or the like.

In one set of embodiments, a factorial design may be used to designate experimental protocols from the experiment framework. Factorial designs suitable for use with the present invention include those available in the literature, for example, factorial designs that use every single permutation and/or combination of factors, or only a subset thereof, as well as the techniques further described herein. The levels within each factor can be selected sequentially or randomly, depending on the factorial design.

In another set of embodiments, the invention provides a method of constrained factorial design. In constrained factorial design, a plurality of factors are selected, each having a plurality of levels. For each experimental protocol, one level is chosen from each of the plurality of factors and applied to the experimental protocol. However, in constrained factorial design, not all of the factors can be independently applied to each experiment. Thus, a level chosen for a first experiment may constrain a level chosen for a second experiment. It should be noted that this method is not only applicable to reaction sites within a single chip, but to other systems having pluralities of groups, each comprising a plurality of elements, at least one group of which contains more than one element.

As an example, multiple experiments may be performed simultaneously in multiple reaction sites within a single chip. Some factors may apply to each individual reaction site within an chip independently of the other reaction sites within the chip, while other factors apply to all of the reaction sites within the single chip simultaneously. A non-limiting example of a factor that can be independently applied to each reaction site is the concentration of a substance, i.e., the concentration of a substance in a first reaction site is independent of the concentration of the substance in a second reaction site (including zero concentration). A non-limiting example of a factor that simultaneously affects each reaction site within an chip, and cannot typically be independently determined for each reaction site within the chip, is temperature.

Thus, one set of embodiments of the invention provides methods for generating a plurality of experimental protocols, using constrained factorial design, such that each chip within the experimental protocol is affected by a set of factors that affects the entire experiment. Within each chip, each reaction site may have the same or different individual factors, i.e., which independently affect each reaction site. In some cases, one or more reaction sites within an chip may be left undefined, e.g., as shown by the example below.

Thus, one set of embodiments of the invention provides methods for generating a plurality of experimental protocols, using constrained factorial design, such that each chip within the experimental protocol is affected by a set of factors that affects the entire experiment. Within each chip, each reaction site may have the same or different individual factors, i.e., which independently affect each reaction site. In some cases, one or more reaction sites within an chip may be left undefined, e.g., as shown by the example below.

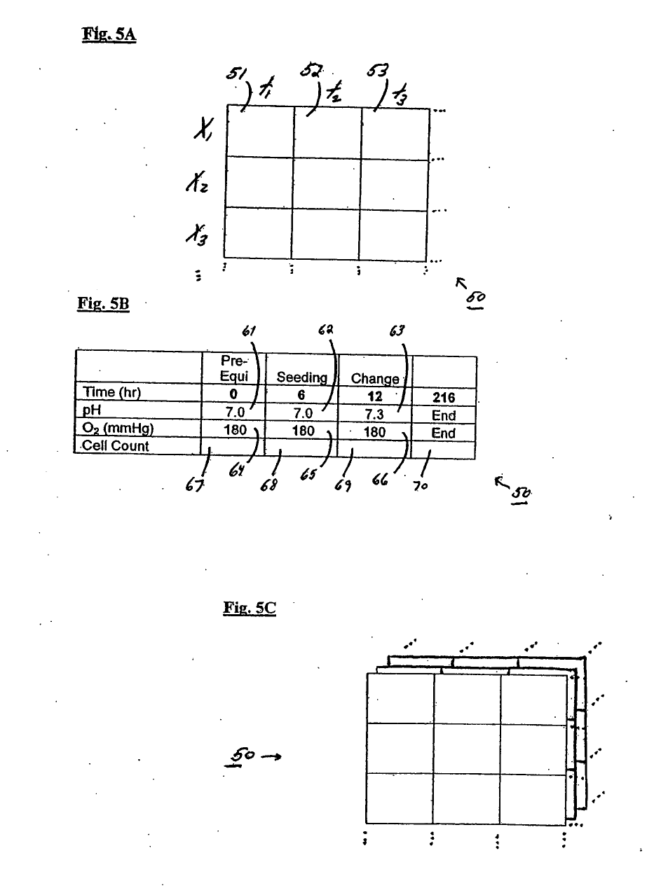

As a non-limiting example of a constrained factorial design, with reference to FIG. 4A, a series of factors A, B, C, and D are to be applied in an experimental framework, each with several levels—i.e., A₁-A₄, B₁-B₃, C₁-C₅ and D₁-D₄. It should be understood that more or fewer levels may be independently assigned to each particular factor, depending on the actual application, and that the number of factors may differ, as well. As a particular non-limiting example, if factor A represents temperature, and there are four temperatures to be tried, then each of the four temperatures will be assigned to one of the four levels (A₁-A₄) in this example.

A factorial design is then applied in FIG. 4B to create a series of experimental protocols (corresponding to the rows) within an experimental framework 55, in each of which the level of each factor is are randomly applied (although, in other factorial designs, the levels may be systematically altered, instead of randomly altered). Thus, experimental protocol P1 may have levels A₁, B₁, C₂, D₂, experimental protocol P2 may have levels A₁, B₃, C₂, D₄, etc.

In this example, the experimental protocols may be applied to experiments on a chip 50 that has an array of two reaction sites 51, 52, as shown in FIG. 4C. In this example, factor A affects the entire chip (for example, ambient temperature), while factors B, C, and D each affect only a single reaction site within a chip (for example, concentration of various reagents for a chemical reaction, etc.). While experimental protocols P1 and P2 in this example can each be assigned to a chip alpha (α), as shown in FIG. 4D, experimental protocols P3 and P4 cannot be each assigned to a common chip, as levels A₂ and A₃ cannot be applied simultaneously to the common chip as they denote two different temperature conditions which are mutually exclusive. However, when the experimental protocols are then sorted to group like levels of “global” factor A together, as shown in FIG. 4D, it will be seen that some protocols may be grouped. However, not every reaction site within each chip is necessarily assigned an experimental protocol. For instance, as only one experiment is required at level A₃, the chip to which experimental protocol P4 has been assigned (chip gamma (γ)) does not contain a second experimental protocol. Similarly, experimental protocol P6, which requires level A4, has been assigned to chip delta (δ), which also does not contain a second experimental protocol. In particular, experimental protocols P4 and P6 cannot be combined on a single chip, as different levels A₃ and A₄ (e.g., temperature) would be required, and these factors cannot be simultaneously be applied to a single chip. Yet, protocols P3 and P5 may be assigned to a same group, beta (β).

In some cases, an experimental protocol as created by the factorial input then may be expressed and recorded in a data structure representation not by the actual values of the factor levels but, rather, as differences with respect to a baseline (i.e., starting) experimental condition. For instance, if a factor X is to be set to a value Xₜ at a time “t,” then the set point value of the factor may be recorded as (Xₜ-X₀), where X₀ is the baseline value, rather than Xₜ. As a specific example, if the factor is temperature and the baseline temperature is 25° C., a value of 20° C. may be recorded as −5° C. (20° C. to 25° C.) rather than 25° C. This approach, rather than storing the temperatures themselves or the increment in temperature from one level to the next is very useful in several respects. It preserves the integrity and reproducibility of the experimental sequence that produced specific data. At the same time, it provides a way that a subsequent experiment can be run, varying the conditions around a particular point in the experiment, without rendering meaningless all of the data subsequent to that from the point being considered. It also facilitates more compact storage of the data representing the experimental protocols. As an example, if temperature were to be varied in steps from 26° C. to 28° C. to 31° C. to 33° C., and it were then desired to explore more carefully the region around 29° C., one could do so by adding a set point at 29° C. without rendering meaningless or unclear the data at higher temperatures.

This approach may be implemented, for example, by storing an experimental protocol (for example, a cell culture protocol) in a matrix (more precisely, in a data structure corresponding to a matrix) having one row per experimental protocol and one “delta value” in each matrix cell (i.e., memory location for the cell). The matrices may also be translated into a program that can be executed on an automated device, for example, a cluster tool-type device and/or an automated cell culture device. The program, when executed, causes the device to perform the entire experimental framework and/or one or more experimental protocols within the experimental framework.

In one aspect of the invention, one or more experimental protocols within an experimental framework may then be mapped to one or more reaction sites and/or one or chips. In addition, in some cases, the experimental protocols may also be converted to a series of sequential operations that will achieve the factorial design of the experimental framework. In certain cases, each experimental protocol may be assigned an experiment number or tag.

As an example, in one set of embodiments, one or more experimental protocols are used to define a descriptor, which can then may be used to control a system, such as a cluster tool-type device and/or an automated cell culture device, e.g., as described above. For instance, data within the descriptors may be used to operate one or more handling devices within the system. The descriptor thus may define experimental protocols in which one or more factors changes in time, for example, at a first time, a factor may change, and at a second time, the factor and/or a different factor may change. The change may be, e.g., a step change or a gradual change from one level to a new level. The descriptor is thus able to include such time-based information.

The descriptors may be converted into a series of commands to be sent to an automated system using a suitable computer program (e.g., a parser or interpreter or translator) or dedicated hardware internally or externally to the automated system, the commands being such that, when executed, they direct the automated system to perform the experimental protocol(s) encoded by the descriptor. The actual series of commands will depend on the application, and will vary based on factors such as the system configuration, the experimental framework, and the signals and programming language(s) used. For example, a system may comprise one or more storage modules, one or more temperature or incubation modules, one or more addition modules, one or more sampling modules, etc., as previously described, and the computer program may convert the descriptors into a series of commands that are sent to the system. In some embodiments, the computer may control operation of the system and send the appropriate commands at the appropriate times; in other embodiments, the system itself may be able to function autonomously once the descriptor has been introduced into the system.

As a first example, if a system comprises an incubation module, the descriptor may designate that a chip and/or a reaction site be stored under a first condition (e.g., temperature, degree of agitation, etc.) at a first point of time, and at a second condition at a later point of time. The program then converts the descriptor into commands that are sent to the system at the appropriate times, directing the incubation module to present the appropriate condition at the appropriate times. As a second example, if a system comprises a handling device, a storage module, and a fluid transfer module, the descriptor may designate that a chip and/or a reaction site be filled with a first fluid at a first point of time, and be filled with a second fluid at a second point of time. The program then converts the descriptor into commands that are sent to the system at the appropriate times, e.g., directing the handling device to move a chip to the fluid transfer module at the first point of time, directing the fluid transfer module to fill the chip and/or the reaction site, then directing the handling device to move the chip to the storage module; and at a second point of time, directing the handling device to move the chip from the storage module to the fluid transfer module, directing the fluid transfer module to empty the chip and/or the reaction site, directing the fluid transfer module to fill the chip and/or the reaction site with the second fluid, then directing the handling device to move the chip to the storage module. Those of ordinary skill in the art will be able to adapt the program as necessary if other modules are used, e.g., heating modules, cooling modules, identification modules, measurement modules, assay modules, etc., e.g., as previously described.

In another aspect, the present invention also provides systems and methods for recording data obtained from experimental protocols such as those described above, and storing them using the associated “descriptor” or other label tied to the experiment, i.e., a representation in a machine- or computer-readable medium of the factors and levels of the experimental protocol which produced the data. That is, data obtained from an experimental protocol can be labeled with a “descriptor,” which is used to identify or “tag” the data, e.g. for future analysis, for comparison with other data (e.g., from the same or different experimental protocols), to show regulatory compliance, etc. The data may be stored within a database, and optionally searched using the descriptor. In some cases, the data within the descriptor is used for controlling one or more experiments, e.g., in conjunction with an automated cell culture device or other automated device, for example, but not limited to, those described in U.S. patent application Ser. No. 10/863,585, filed Jun. 7, 2004, entitled “System and Method for Process Automation,” by Rodgers, et al., published as U.S. Patent Application Publication No. 2005-0037485 on Feb. 17, 2005, incorporated herein by reference. In other cases, the descriptor is used for recording data from one or more experiments. In still other cases, the descriptor may be used for both controlling one or more experiments and for recording data therefrom. In yet other cases, an experimental protocol and data resulting from an experiment based on the experimental protocol may be “tagged” or linked via a descriptor. Or separate tags may be used for the protocol and the data, and a correspondence table maintained in its own memory locations, available only to authorized parties. In that way, an unauthorized party will not be able to correlate experimental design and results.

In some cases, the descriptor may also include time-based information. For example, the descriptor may include a matrix in which each row (or column) represents an individual factor (e.g., temperature or concentration), while each column (or row) represents an instant in time (for example, as a well-defined series of time events, as instants in time where one or more factors were changed, etc.). Non-limiting examples of factors which may be represented as values within the descriptor include temperature, concentration of a gas (e.g., O₂, CO₂, N₂, air, etc.), pH, concentration of a nutrient, cell viability (if cells are present), etc. The matrix may have any number of rows and, independently, any number of columns, depending on the complexity of the experiment. In some cases, one row of the matrix is used to identify the columns, i.e., the factors of the descriptor, for instance, a row of the matrix may be designated as O₂, CO₂, N₂, pH, cell viability, etc.

The descriptor is not limited to a representation of individual factors versus time. Other variables may be used as the independent variable besides time, such as, for example, concentration of a nutrient, concentration of a product, pH, cell density, etc. Thus, as an example, a descriptor may comprise a matrix which includes (in addition to data which defines the meaning of each row and column) a row (or column) representing an individual factor (e.g., temperature or concentration), and each column (or row) representing a certain threshold event (for example, an event where a certain concentration of a substance is reached, where a certain cell density is reached, or the like).

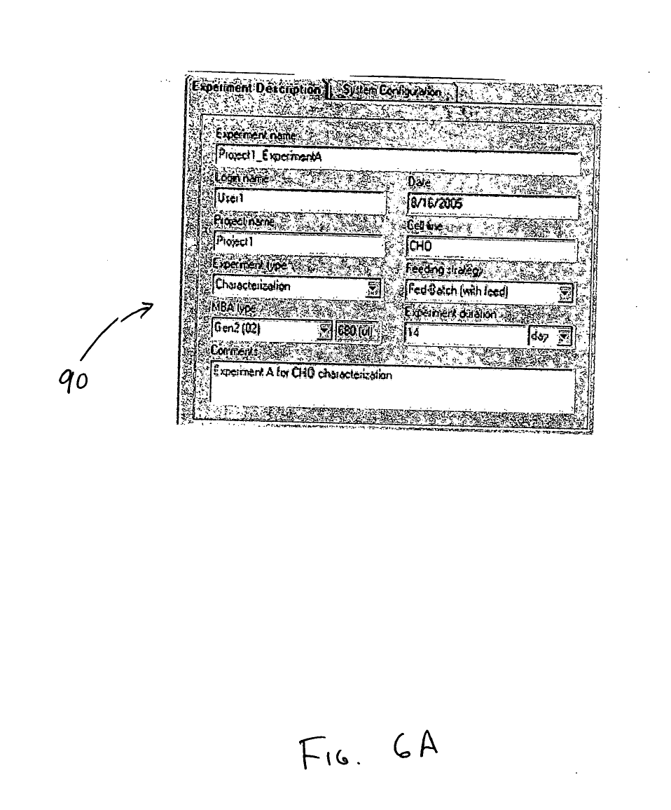

Examples of descriptors for experimental protocols are shown in FIG. 5. In FIG. 5A, descriptor 50 is a matrix having a plurality of rows and a plurality of columns. In this matrix, column 1 represents time t₁ (for instance, the beginning of an experiment), column 2 represents time t₂, column 3 represents time t₃, etc. Similarly, Row 1 represents a first factor X₁, row 2 represents a second factor X₂, row 3 represents a third factor X₃, etc. The values in each row may be the same or different, depending on the experimental protocol. Thus, for example, in FIG. 5A, location 51 (representing the value of a first factor at time t₁) may have a first value, while location 52 may have a different value (e.g., representing a change that occurs in first factor X₁ at time t₂, and location 53 may have a value that is the same or different than location 51 or location 52, etc. It should be noted that not all factors within the descriptor need to be changed during the experimental protocol, i.e., in some cases, the values stored in the locations of one or more rows of the descriptor in FIG. 5A may all be the same. The values within the locations may be absolute values, or relative (offset) values, e.g., location 51 may represent an absolute value, while locations 52, 53, etc., may store a number that represents an offset, which is to be added to a reference value or to the previous value (thus, a value of “0” would mean that the factor does not change). In addition, in some cases, a descriptor may be prepared in which the rows and columns are reversed, i.e., the columns of the descriptor may represent factors, while the rows of the descriptor represent time. A two-dimensional descriptor matrix may, of course, be converted to an equivalent one-dimensional array representation. Such a conversion might be done to serialize a data stream for parsing to generate commands to an automated apparatus.

A specific, non-limiting example of a descriptor useful in cell culture experiments is shown in FIG. 5B. In this figure, within descriptor 50, column 1 (designated as a “pre-equilibrium” step) represents time 0 h, column 2 (designated as a “seeding” step) represents time 6 h, column 3 (designated as a “change” step) represent time 12 h, and column 4 represents the end of the experiment at time 216 h (i.e., 9 days). Row 1 represents the time (in hours), row 2 represents the pH, row 3 represents oxygen (O₂) content (in mmHg), and row 5 represents the cell count. In this experimental protocol, the pH remains at 7.0 for the pre-equilibrium and seeding steps (61 and 62, respectively), then is raised to 7.3 at the change step 63 (i.e., after 12 h). In contrast, the oxygen content is held steady at 180 mmHg throughout the entire experimental protocol (64, 65, 66).

Also in FIG. 5B, it should be noted that the row labeled “Cell Count” has not been filled in (67, 68, 69, 70). Instead, these locations are used for data storage of the experiment. For example, at times 0 h, 6 h, 12 h, and 216 h, the cell count of the experiment may be determined (e.g., using optical or electronic techniques), and such data may be stored within descriptor 50.

In some embodiments, a third dimension may also be added to the descriptor, for example, to store the results of a series of experiments. As an example, descriptor 50 in FIG. 5C is composed of a series of layers, each with a series of rows and columns. Each layer within FIG. 5C represents a different experiment. For instance, each layer may represent a replicate of the same experimental protocol and/or a different experimental protocol.

The descriptors in FIG. 5 are meant to be explanatory in nature and not limiting. Those of ordinary skill in the art will be able to add or modify the rows and/or columns as necessary to a descriptor, depending on the specific application(s). For instance, the descriptor may have more or fewer columns, depending on the particular experiment, or additional rows could be added representing CO₂ concentration, N₂ concentration, relative humidity, concentrations of nutrients (e.g., glucose, glutamine, pyruvate, and/or various amino acids, vitamins, hormones, serum, ions, or the like), temperatures, pressures, cell types, cell densities, cell cycle positions, cell dimensions, substrates, shear rates, or the like, e.g., as previously described.

Moreover, the descriptors as disclosed herein are not limited to cell culture experiments, but more generally can be used in any experiment (or series of experiments) where at least one factor is changed during the experiment. For instance, a descriptor may be prepared for a chemical reaction (or series of chemical reactions); as an example, the descriptor may include one or more times, and rows representing reagent concentrations, reaction conditions, product concentrations, or the like.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising acts of:
culturing a plurality of cell cultures using an automated cell culture device;
for each cell culture, operating the automated cell culture device to collect data representative of a plurality of experimental factors of the cell culture at a plurality of times, using sensors appropriate for the desired data; and
from the automated cell culture device, automatically recording the data in a data structure in a computer-readable data store, the data structure comprising, for each culture, fields in a computer-readable memory defining a matrix addressable in two dimensions, a first dimension representing experimental factors and a second dimension representing time events.

2. The method of claim 1, wherein the act of recording the data includes recording the data within the data structure as a deviation from a baseline condition.

3. The method of claim 1 or claim 2, wherein the act of culturing a plurality of cell cultures comprises directing a robot to manipulate at least some of the plurality of cell cultures to effect a change in one or more of the experimental factors.

4. The method of claim 3, wherein the act of culturing a plurality of cell cultures comprises culturing a plurality of cell cultures on a plurality of reaction arrays, one or more of which comprises more than one location suitable for culturing cells.

5. The method of claim 1, wherein the act of culturing a plurality of cell cultures comprises culturing a plurality of cell cultures on a plurality of reaction arrays, one or more of which comprises more than one location suitable for culturing cells.

6. The method of claim 4 or claim 5, wherein the act of culturing a plurality of cell cultures on one or more reaction arrays comprises mapping the cell cultures to the reaction arrays to minimize a number of reaction arrays required to perform the cell cultures.

7. The method of any one of claims 1, 2, or 5, further comprising generating the plurality of cell cultures using a factorial design.

8. A computer-implemented method for use in performing cell culture experiments, comprising acts of operating a computer to:
present to a user an interface for receiving a set of experimental factors, one or more levels for each experimental factor, and one or more times at which one or more experimental factor values are to be set and/or one or more measurements are to be taken;
receive, as input from the user, the set of experimental factors and the one or more times;
create a data structure corresponding to the received one or more experimental protocols, using the received factorial design, each experimental protocol comprising one or more values for the experimental factors and one or more times; and
assign each experimental protocol to a specific cell culture contained in a reactor array.

9. The method of claim 8, wherein the data structure comprises fields defining a matrix addressable in two dimensions, a first dimension representing the experimental factors and a second dimension representing time.

10. The method of claim 8, further comprising issuing commands to an automated cell culture device, which commands, when acted upon by the device, cause the device to perform at least some of the experimental protocols on one or more reactor arrays.

11. The method of claim 10, wherein the commands are issued in response to reading the data structure.

12. The method of claim 10, further comprising the automated cell culture device performing at least some of the experimental protocols on cell cultures contained within one or more reactor arrays.

13. The method of claim 10, wherein the automated cell culture device comprises a robot able to manipulate at least some of the reactor arrays to move them in order to effect at least some actions of one of the experimental protocols.

14. The method of claim 13, wherein the robot is able to rotate a reactor array about an axis and/or translationally move a reactor array in at least one of a direction substantially perpendicular to the axis and a direction substantially parallel to the axis.

15. The method of claim 13, wherein the robot is able to move a reactor array from a first module that subjects the reactor array to a first condition, to a second module that subjects the reactor array to a second condition different from the first condition.

16. The method of claim 10, further comprising the automated cell culture device performing more than one experimental protocol to a plurality of cell cultures contained in a single reactor array.

17. The method of claim 10, further comprising the automated cell culture device performing a single experimental protocol to each of a plurality of cell cultures contained in a single reactor array.

18. The method of claim 10, further comprising collecting data from the cell cultures contained within one or more reactor arrays as part of performing the experimental protocols.

19. The method of claim 18, comprising assigning at least some of the collected data to the data structure.

20. The method of claim 8, wherein the act of creating a data structure comprises creating the data structure using a constrained factorial design.

21. The method of claim 8, wherein assigning each experimental protocol comprises:
providing a plurality of reactor arrays; and
assigning experimental protocols to specific cell cultures contained within the plurality of reactor arrays such that a minimal number of reactor arrays are used.

22. An article, comprising:
a machine-readable medium having a program stored thereon, which program comprises instructions for, when executed, causing a computer-driven system to perform acts of:
defining a plurality of experimental factors for a cell culture;
for at least some experimental factors, defining a plurality of levels;
generating a plurality of experimental protocols, using, for each factor, a respective level selected from the plurality of levels; and
for each experimental protocol, applying the experimental protocol to a cell culture using an automated cell culture device.

23. A method, comprising acts of:
providing a plurality of reactor arrays, each comprising a plurality of reactors;
defining at least one reaction factor that may, if selected in an experimental protocol, independently operate on a selected reactor;
defining at least one array factor that simultaneously, but not independently, operates on each reactor within a single reactor array;
for each reaction factor and each array factor, defining a plurality of levels;
generating one or more sets of experimental protocols applicable to the plurality of reactor arrays such that at least one set of the one or more sets includes (1) a first experimental protocol applicable to a first reactor of a particular reactor array selected from the plurality of reactor arrays, and (2) a second experimental protocol applicable to a second reactor of the particular reactor array, the first experimental protocol and the second experimental protocol each being generated using, for each selected reaction factor and each selected array factor, a respective level selected from the plurality of levels, wherein the first experimental protocol includes a first level of a reaction factor, and the second experimental protocol includes a second level of a reaction factor that is different from the first level; and
applying the set of experimental protocols to the corresponding particular reactor array using an automated device.

24. The method of claim 23, wherein the act of generating one or more sets of experimental protocols is implemented using a computer.

25. The method of claim 23, wherein at least one reactor comprises a cell culture.

26. The method of claim 25, wherein at least one reaction factor includes cell viability.

27. The method of claim 25, wherein at least one reaction factor includes concentration of a cell nutrient.

28. The method of claim 23, wherein at least one reaction factor includes cell type.

29. The method of claim 23, wherein at least one reaction factor includes pH.

30. The method of claim 23, wherein at least one reaction factor includes concentration of a species.

31. The method of claim 23, wherein at least one reaction factor includes concentration of a dissolved gas.

32. The method of claim 23, wherein at least one array factor includes temperature.

33. The method of claim 23, wherein at least one array factor includes concentration of a gas.

34. The method of claim 23, wherein the act of generating comprises generating one or more sets of experimental protocols using a computer.

35. The method of claim 23, wherein the automated device comprises a robot able to manipulate the particular reactor array.

36. The method of claim 35, wherein the robot is able to rotate a reactor array about an axis and/or translationally move a reactor array in at least one of a direction substantially perpendicular to the axis and a direction substantially parallel to the axis.

37. The method of claim 35, wherein the robot is able to move a reactor array from a first module that subjects the reactor array to a first condition, to a second module that subjects the reactor array to a second condition different from the first condition.

38. The method of claim 23, further comprising collecting data from the particular reactor array.

39. The method of claim 38, further comprising recording the data in a data structure in a data store comprising fields defining a matrix addressable in two dimensions, a first dimension representing experimental factors and a second dimension representing time events.

40. The method of claim 38, further comprising recording the corresponding experimental protocol to the data structure.

41. The method of claim 38, wherein the act of recording the data includes recording the data within the data structure as a deviation from a baseline condition.

42. An article, comprising:
a machine-readable medium having a program stored thereon, which program comprises instructions for, when executed, performing acts of:
providing a plurality of reactor arrays, each comprising a plurality of reactors;
defining at least one reaction factor that may, if selected in an experimental protocol, independently operate on a selected reactor;
defining at least one array factor that simultaneously, but not independently, operates on each reactor within a single reactor array;
for each reaction factor and each array factor, defining a plurality of levels;
generating one or more sets of experimental protocols applicable to the plurality of reactor arrays such that at least one set of the one or more sets includes (1) a first experimental protocol applicable to a first reactor of a particular reactor array selected from the plurality of reactor arrays, and (2) a second experimental protocol applicable to a second reactor of the particular reactor array, the first experimental protocol and the second experimental protocol each being generated using, for each selected reaction factor and each selected array factor, a respective level selected from the plurality of levels, wherein the first experimental protocol includes a first level of a reaction factor, and the second experimental protocol includes a second level of a reaction factor that is different from the first level; and
applying the set of experimental protocols to the corresponding particular reactor array using an automated device.

43. An apparatus, comprising:
an automated cell culture device comprising the article of claim 42.

44. The apparatus of claim 43, wherein the automated cell culture device comprises a robot able to manipulate reactor arrays in order to effect at least some actions of one or more of the set of experimental protocols.

45. A computer-implemented method, comprising an act of operating a computer to:
prompt a user to input:
a plurality of groups, each comprising a plurality of elements, at least one group containing more than one element;
at least one reaction factor that may, if selected in an experimental protocol, independently operate on a selected element;
at least one group factor that simultaneously, but not independently, operates on each element within a single group;
for each elemental factor and each group factor, a plurality of levels;
generate one or more sets of protocols applicable to the plurality of groups such that at least one set of the one or more sets includes (1) a first protocol applicable to a first element of a particular group selected from the plurality of groups, and (2) a second protocol applicable to a second element of the particular group, the first protocol and the second protocol each being generated using, for each selected elemental factor and each selected group factor, a respective level selected from the plurality of levels, wherein the first protocol includes a first level of an elemental factor, and the second protocol includes a second level of an elemental factor that is different from the first level;
issue the one or more sets of protocols to an automated device; and
cause the automated device to apply the one or more sets of protocols to an experimental system comprising a plurality of discrete experiments.

46. The method of claim 45, wherein each of the plurality of discrete experiments comprises a biological organism upon which an experimental protocol is performed.

47. An article, comprising:
a machine-readable medium having a program stored thereon, which program comprises instructions for, when executed, performing acts of:
presenting to a user an interface for receiving a list of experimental factors, one or more levels for each experimental factor, and one or more times at which one or more experimental factor values are to be set and/or one or more measurements are to be taken;
receiving, as input, the list of experimental factors and the one or more times;
creating a data structure corresponding to the received one or more experimental protocols, using the received factorial design, each experimental protocol comprising one or more values for the experimental factors and one or more times; and
assigning each experimental protocol to a specific cell culture contained in a reactor array.

48. The article of claim 47, wherein the data structure comprises fields defining a matrix addressable in two dimensions, a first dimension representing the experimental factors and a second dimension representing time.

49. The article of claim 47, wherein the machine-readable medium comprises instructions for creating the data structure using a constrained factorial design.

50. The article of claim 47, wherein the machine-readable medium comprises instructions for:
providing a plurality of reactor arrays; and
assigning experimental protocols to specific cell cultures contained within the plurality of reactor arrays such that a minimal number of reactor arrays are used.

51. A method of screening cell culture experiments comprising:
collecting in a data store a plurality of descriptors of automated cell culture experiments which have been performed, and resulting experimental data; and
operating a computer to search the data store for descriptors matching provided descriptor criteria.

52. The method of claim 51 wherein collecting descriptors and experimental data includes storing said descriptors and experimental data separately.

53. The method of claim 51 wherein collecting descriptors includes aggregating from multiple sources descriptors of experiments conducted by those sources.

54. The method of claim 51 wherein operating a computer to search includes operating a computer to search only descriptors for which the searcher is authorized.

55. The method of claim 51 further including charging a fee for searching the data store.

56. A computer-readable medium having recorded thereon a descriptor usable for defining an experiment to an automated cell culture device and encoding desired experimental conditions for a cell culture experiment.

57. A method of performing cell culture experiments comprising recording as a descriptor in a machine-readable form the parameters and specifications for performing an experiment, in a format usable, directly or indirectly, by an automated cell culture device.

58. The method of claim 57 further including recording measurements from said experiment in a machine-readable form which associates the measurements with a corresponding descriptor for the experiment.

59. The method of claim 57, further comprising using an interpreter program, or parser, to interpret a set of one or more related descriptors as a set of desired experimental conditions and generate a corresponding sequence of commands to direct an automated system to execute corresponding experiments in bioreactors manipulated by the automated system.

60. The method of claim 58 wherein the bioreactors are arranged in arrays and the method further includes mapping experimental parameters to the arrays to minimize the number of arrays required by an experiment.

61. A method of performing a factorial or multi-factorial experimental design including operating a computer to assist a user in the creation of a set of machine-readable descriptors corresponding to said factorial design.

62. The method of claim 57 further including using the descriptors as “tags” to index data resulting from the experiment for later search and analysis

63. A method of facilitating efficient cell culture experimenting comprising searching previous cell culture experimental results recorded in a data store by comparing tags of data sets in said store and returning results ranked by degree of similarity to the query tag.

64. A computer-readable medium having recorded thereon signals defining operations for performing the method and/or constituting the apparatus of any of the foregoing claims, when executed on a processor.
